(19) United States
(12) Patent Application Publication
(10) Pub. No.: US 2019/0099109 A1
(43) Pub. Date: Apr. 4, 2019

(54) PORTABLE DEVICE FOR MONITORING A PLURALITY OF KETONES AND RELATED METHOD

(71) Applicant: Invoy Technologies, LLC, Aliso Viejo, CA (US)

(72) Inventor: Lubna Ahmad, Chandler, AZ (US)

(21) Appl. No.: 15/665,352

(22) Filed: Jul. 31, 2017

Related U.S. Application Data
(60) Provisional application No. 62/368,311, filed on Jul. 29, 2016.

(51) Int. Cl. A61B 5/08; A61B 5/00; A61B 5/097; A61B 5/145

(52) U.S. Cl. CPC A61B 5/082; A61B 5/6822; A61B 5/681; A61B 5/097; A61B 5/14532; A61B 5/002; A61B 5/742; A61B 5/7405; A61B 5/14546

(57) ABSTRACT

Systems and methods of and for portable device for monitoring a plurality of ketones. A system may include a device comprising a first ketone body measurement unit that measures a concentration of a first ketone body, a second ketone body measurement unit different from the first ketone body measurement device that measures a second ketone body different from the first ketone body, and an output device that receives the first and second ketone body concentration measurements and presents information to the user relating to the first and second ketone body concentration measurements, optionally simultaneously. The device may be portable. The method may include using a portable device to obtain a breath acetone concentration measurement of a user and a blood ketone body concentration measurement comprising at least one of an acetoacetate concentration and a β-HBA concentration of the user. The method may also include various other steps.

### INCORPORATION BY REFERENCE TO ANY PRIORITY APPLICATIONS

This application claims priority benefit of U.S. Provisional Application No. 62/368,311 (Dkt. No. INVOY.025A), filed on Jul. 29, 2016. All of the above applications are incorporated by reference herein and are to be considered a part of this specification. Any and all applications for which foreign or domestic priority claim is identified in the Application Data Sheet as filed with the present application are hereby incorporated.

### BACKGROUND

### Field

The present application relates to apparatus, systems, and methods for analyzing ketone bodies and for providing information to a user about relative changes in those ketone bodies. Some embodiments disclosed herein may be useful in providing such information to a user in the context of a relatively small or portable apparatus or system that is suitable for point of care or home use.

### Description of the Related Art

As production, consumption, conversion and dynamic equilibrium of ketone bodies are studied, methods and devices are needed to facilitate understanding and use of relationships and discoveries. There is a need, therefore, for such methods and devices.

This need is also impacted by the general trend toward the use of “wearables” with respect to consumer electronics and certain types of health-related devices. Examples include wrist watch devices that include such things as heart rate monitors, blood pressure monitors, and the like. The term “wearable” is used herein according to its commonly understood meaning, and includes, among other things, devices that the user wears on some part of his or her body for relatively extended durations, for example, such as for hours or days, as one might wear a wrist watch. With respect to health-related devices that perform periodic tests, the wearable normally could be worn for periods that extend beyond the specific time actually required to perform the test, although this is not necessarily the case.

Wearables may be particularly useful or beneficial for applications in which the wearable is used for a health-related test that is performed multiple times in a relatively short period of time. One example is a diet program. Acetone as a breath analyte has been correlated with fat metabolism. Because a primary goal of many dieters involves burning fat to lose weight, such dieters may use acetone breath analysis devices to measure their fat burn rate, e.g., as a measure of the effectiveness of their weight loss program. Other examples in which regular health-related measurements might be taken include exercise programs or heavy physical activity.

Currently, there are few, if any, commercially-available or known wearable breath analysis devices. Many breath analysis devices are laboratory or table top devices. Those that are portable generally are neither wearable nor readily adaptable for wearability. Moreover, in many of these types of applications, the user is engaged in another activity besides the health-related test, e.g., such as may be the case for exercises or physical activities. The user's hands thus may be occupied with other tasks and unavailable to operate the breath analysis device, or it may be very inconvenient to do so. Further, in some breath analysis applications, such as where the user is prostrate and a nasal cannulas has been installed, the user may be physically unable to operate the breath analysis device. Such users often have limited gas or breath flow and no possibility of forced expiration.

### SUMMARY

To address these limitations and advance the art, a device is provided that comprises a first ketone body measurement device that measures a concentration of a first ketone body, a second ketone body measurement device different from the first ketone body measurement device that measures a second ketone body different from the first ketone body, and an output device that receives the first and second ketone body concentration measurements and presents information to the user relating to the first and second ketone body concentration measurements, optionally simultaneously. The device may be portable. In some embodiments, the output device is detached or spaced from the first and second ketone body measurement devices and comprises a mobile device, e.g., a smart phone, tablet, or the like.

The ketone sample may be obtained directly from the user at the point of care or user's location and may be used in the measurement devices described herein, even if the sample must be moved to or from the sample procurement location (e.g., from the user's finger to the test strip and test device as described in connection with the embodiment of FIG. 16). This is opposed to devices in which the sample is drawn but then transported to a central lab or processing facility away from the point of care or user's location. The “user” as the term is used herein may refer to the person whose ketone body samples are being obtained and measured. This may or may not be the same person who actually conducts the measurements or observes/evaluates the outputs. In the illustrative embodiments and methods described herein, the user may be referred to as being both the person from whom the samples are obtained and the person operating the devices or conducting the measurements, although this need not necessarily be the case in all instances.

In some embodiments, a device is provided that comprises a breath acetone measurement device that measures a breath acetone concentration of a user, a blood ketone body measurement device that measures at least one of a blood acetoacetate concentration and a blood β-HBA concentration of the user, and an output device that receives the breath acetone concentration measurement and the at least one of the blood acetoacetate concentration and the blood β-HBA concentration and presents information to the user relating to these concentration measurements, optionally simultaneously. This device may be portable. In some embodiments, the output device is detached or spaced from the measurement devices and comprises a mobile device.

In some embodiments, a method is provided that comprises providing a portable device that comprises a first measurement device and a second measurement device different from the first measurement device, using the first measurement device to obtain a concentration measurement of a first ketone body in a user, using the second measurement device to obtain a concentration measurement of a second ketone body different from the first ketone body in the user, and using the device to output information relating to the first and second ketone body concentration measurements, optionally simultaneously. In some embodiments of the method, the output is displayed on a mobile device.

In some embodiments, a method is provided that comprises providing a portable device that provides an output, using the portable device to obtain a breath acetone concentration measurement of a user, using the portable device to obtain a blood ketone body concentration measurement comprising at least one of an acetoacetate concentration and a β-HBA concentration of the user, communicating the breath acetone concentration measurement and the blood ketone body concentration measurement to the output device, and using the output device to provide information to the user.

In some embodiments of the methods disclosed herein, the output may comprise a ratio of two or more ketone body concentration measurements, e.g., breath acetone and at least one of blood acetoacetate concentration and blood β-HBA concentration. The method may further comprise using an alarm to provide notification, e.g., when the ratio reaches a threshold value or range.

### BRIEF DESCRIPTION OF THE DRAWINGS

The accompanying drawings, which are incorporated in and constitute a part of the specification, illustrate various embodiments of systems and methods, and, together with the general description given above and the detailed description given below, serve to explain the principles of various embodiments disclosed herein.

FIG. 1 shows an embodiment of a device comprising a breath acetone measurement device and a blood ketone measurement device.

FIG. 2 shows a user wearing the wearable of FIG. 1 prior to conducting ketone body measurement tests.

FIG. 3 shows the user wearing the wearable of FIG. 1 while conducting a breath acetone concentration measurement test.

FIG. 4 shows a partial cutaway side view of a mouthpiece for the wearable device of FIG. 1.

FIGS. 5A-5B shows a modification of the mouthpiece of FIG. 4 including a bite valve.

FIG. 6 shows a partial cutaway side or angled view of the wearable of FIG. 1.

FIG. 7 shows a perspective view of an electromagnet detection device.

FIG. 8 shows a cross sectional cutaway side view of the device of FIG. 7.

FIG. 9 shows a block diagram of components of the detection device shown in FIG. 7.

FIG. 10 shows a perspective view of various components of the detection device of FIG. 7 disposed about a patient's finger to illustrate use of the device.

FIGS. 11A-11B shows the device of FIG. 7 on the finger of the patient taken along the longitudinal axis of the finger, wherein the arms of the device are shown in cross section.

FIGS. 12A-12F shows a diagram illustrating the affixing of the device of FIG. 7 on the finger of a patient in the course of using and operating the device to make an analyte measurement.

FIG. 13 shows a version of the device of FIG. 7 in which the inner jaws of the device are conformed to the contours of the user's finger.

FIG. 14 shows a side view of the device of FIG. 7 as it is molded to the finger of the user.

FIGS. 15A-15F shows a diagram illustrating the affixing of the device of FIG. 7 on the finger of a patient in the course of using and operating the device to make an analyte measurement.

FIG. 16 shows another embodiment of a device comprising a breath acetone measurement device and a blood ketone measurement device.

FIG. 17 shows an embodiment of a user interface presenting two ketone levels trended over time for one user.

FIG. 18 shows an embodiment of a user interface presenting two ketone levels trended over time for another user.

FIGS. 19, 20A-20B, 21, and 22 show the change in ketone levels for different users, but who are in different physiological states, including pre- and post-exercise states. In particular, subject 7003 is very low carb adapted. These figures also show user interfaces of interest.

### DETAILED DESCRIPTION

Reference will now be made in detail to the various embodiments and methods as described herein and as illustrated in the accompanying drawings, in which like reference characters designate like or corresponding parts throughout the drawings. It should be noted, however, that the invention, in its broader aspects, is not limited to the specific details, representative devices and methods, and illustrative examples shown and described in this section.

The term “ketone bodies” refers to ketones that are present in the body including, without limitation, acetone, acetoacetic acid (also known as acetoacetate), and β-hydroxybutyric acid (also known as β-hydroxybutyrate or β-HBA). Ketone bodies may include endogenous and/or exogenous ketones.

In the case of endogenously produced ketones, such as during fat metabolism, fatty acids are enzymatically converted through B-oxidation to form acetyl-cofactor A (“acetyl-CoA”). The portion of the acetyl-CoA that is not used directly for energy is converted to acetoacetate. Acetoacetate in turn can be converted into β-HBA or acetone. β-HBA is generated from acetoacetate through the enzyme D-β-HBA dehydrogenase. Acetone can be generated from acetoacetate spontaneously or converted enzymatically through acetoacetate decarboxylase.

Once ketone bodies are present in the body, they can be converted. Again, in the case of endogenous ketones, β-HBA, for example, having been created from acetoacetate, can be converted back into acetoacetate. The forward conversion or reaction, (e.g., from acetoacetate to β-HBA), and the reverse conversion or reaction, (e.g., from β-HBA back into acetoacetate) will seek to find and maintain an equilibrium.

The relative concentrations of the respective ketone bodies rarely, if ever, are identical to one another, the concentrations of those ketone bodies routinely change absolutely and relatively with respect to one another. The physiological or pathological implications of these changes have not appeared prominently in the literature.

As a general matter, the concentrations of ketone bodies vary depending on a number of factors, including (e.g., significantly) the extent to which fat is being metabolized. Also as a general matter, the relative concentrations of the individual ketone bodies tend to stay more or less balanced. There are instances, however, in which imbalances may occur. In view of the fact, as noted herein, that relatively little is reported in the literature regarding such imbalances and the causes for them, the present inventors anticipate that their findings in this regard are important mechanisms and phenomenologies. Thus, devices and methods as disclosed herein, which provide the ability to measure a plurality of individual ketones more or less simultaneously, may be of great benefit and, in some cases, could be needed.

During a high state of ketosis, under “normal” circumstances, up to 37% of the acetoacetate produced in the liver can be converted into acetone. Acetoacetate and β-HBA are interconverted by the enzyme beta-hydroxybutyrate dehydrogenase (β-HBA dehydrogenase). This reaction primarily occurs in the mitochondria of liver cells and to a smaller extent in other cells of the body. Under normal circumstances, each of these conversions undergoes forward and reverse reactions so that the tendency is toward a dynamic equilibrium set of concentrations.

The net generation of each of the ketone bodies, however, can vary depending upon a range of factors, and each of the ketone bodies undergoes its own reactions and processes within the body once generated. Accordingly, the relative concentrations of each of the ketone bodies can vary, at times considerably, as a result. Having the ability to measure the concentrations of a plurality of ketone bodies at a given time may offer the capability to understand, assess, and/or explore the underlying phenomenology that is giving rise to differing concentration levels, and, possibly, to understand, assess, and/or explore the associated physiological and/or pathological states within the body.

Accordingly, some embodiments comprise a method in which one uses a device to obtain (e.g., simultaneously or substantially at the same time) a concentration measurement for each of a plurality of ketone bodies. The device outputs the concentration measurements, e.g., simultaneously, on a display, so that comparisons of the respective ketone concentrations can be made. An example of the output is a graphical display that shows one or more ratios, or a comparative plot. In some embodiments, the devices are relatively small, portable devices, each of which is capable of taking concentration measurements of at least two ketone bodies simultaneously or substantially simultaneously.

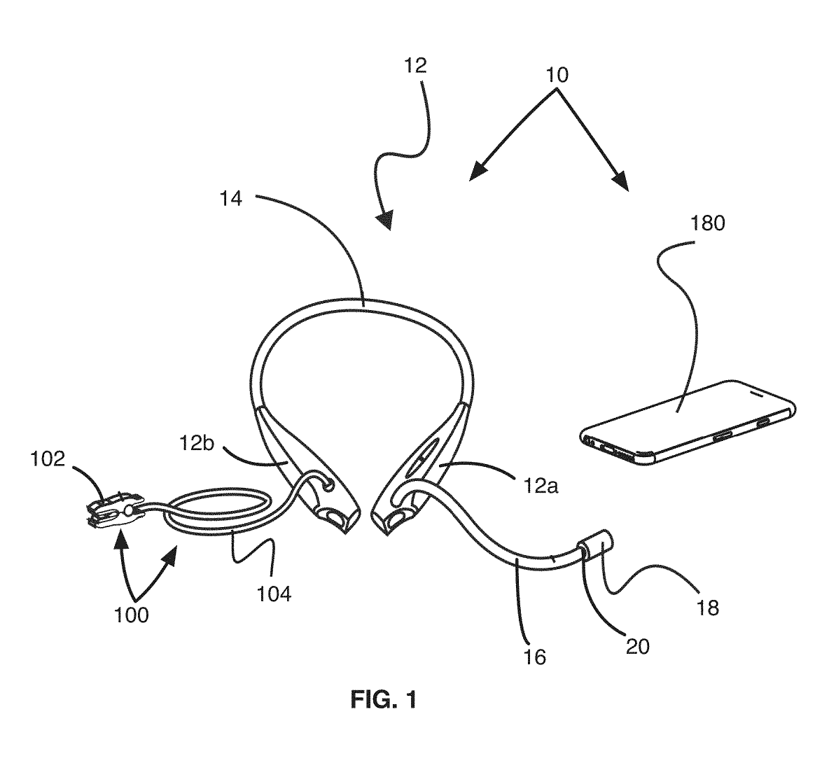

A device 10 according to some embodiments will now be described with reference to FIGS. 1-15. In this example, device 10 is configured to measure the ketone body acetone in the user's or wearer's breath, and at least one of the ketone bodies acetoacetate and β-HBA in the user or wearer's blood.

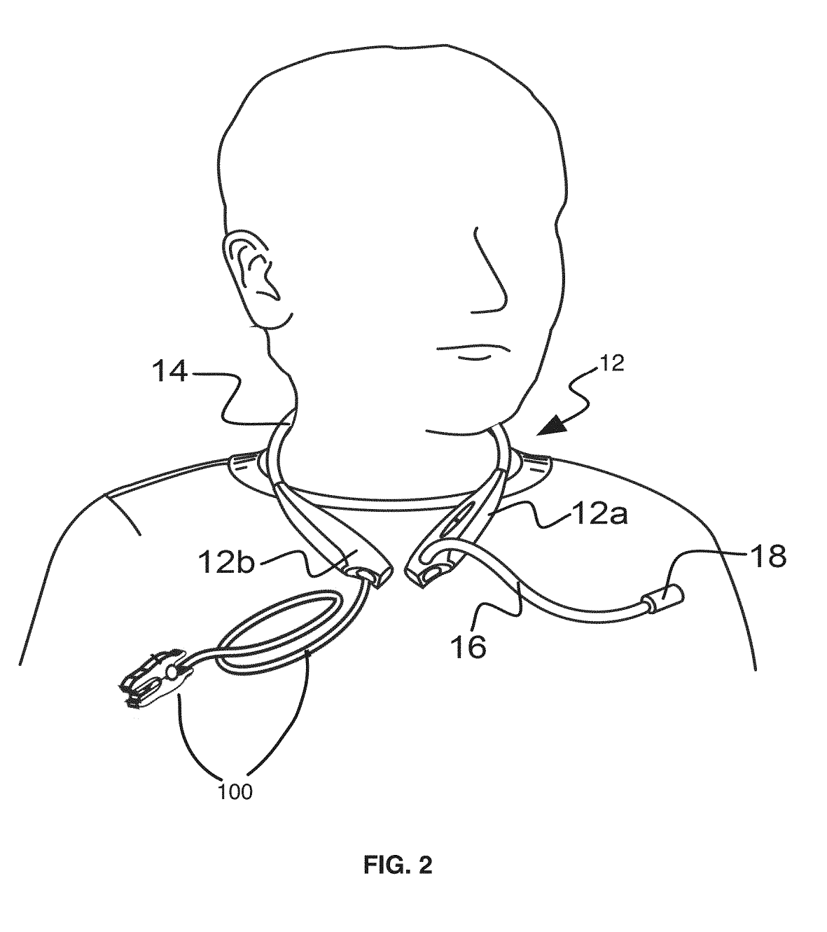

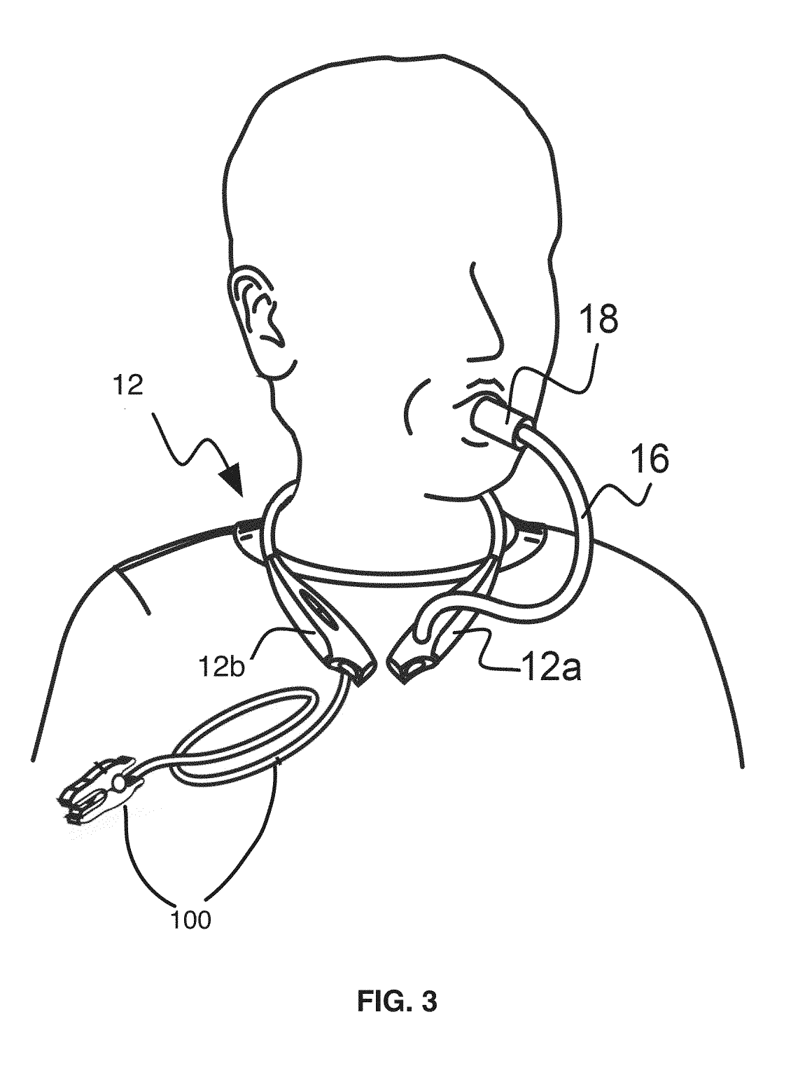

Device 10 comprises a wearable 12 that may be worn and/or used by a user (e.g., detachably worn and/or used). Wearables may take any of a number of forms. Examples include headsets, headset-style devices worn around the neck, devices that may be retained in an arm-mounted or wrist-mounted holster, devices that may be retained in a hip- or waist-mounted holster, and the like. In this embodiment, wearable 12 comprises a headset-style device as shown in FIG. 1 that may be worn around the head or on the neck of a user, e.g., as shown in FIG. 2 (during normal wear but prior to conducting an analyte measurement test) and FIG. 3 (during a breath acetone measurement test). The headset-style device comprises a body that may include a primary body half 12a and a secondary body half 12b. Body halves 12a and 12b are physically connected by a connecting band 14 such as might be found on a commercially-available headset.

Wearable 12 comprises a breath acetone measurement device that comprises a breath collection tube 16 extending from primary body half 12a of wearable 12. A mouthpiece 18 is detachably disposed at the end of the breath tube 16 that is distal with respect to the body half 12a. Breath collection tube 16 in this embodiment comprises a flexible tubing that is substantially impermeable to air or gases, and particularly to the target analyte. The tubing may be relatively inert to breath and components typically found in breath, including the target the analyte.

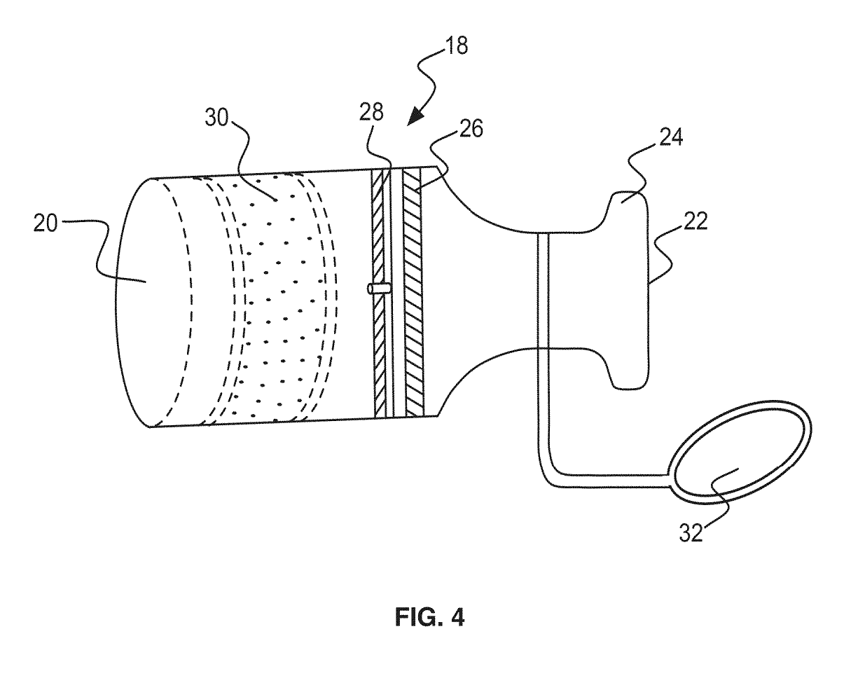

Mouthpiece 18 comprises a skirt 20 at its proximal end with respect to body half 12a for detachably coupling to the distal end of tube 16, as shown in FIG. 4. An input opening 22 is located in mouthpiece 18 at its distal end for receiving a breath sample from the user. A bite collar 24 is disposed on the exterior edge of the mouthpiece 18 at its distal end to aid the user in securing the mouthpiece in his or her mouth. Just inside input opening 22 proximally is a particle filter 26. Moving proximally toward skirt 20, a one-way valve 28 is disposed to allow the breath sample to pass through proximally (toward tube 16 and body half 12a), but to block flow in the reverse direction. Valve 28 may assume a variety of forms, but in this illustrative embodiment it comprises a flapper or butterfly valve. Immediately proximal to valve 28 is a fluid conditioner, which in this embodiment comprises a desiccant filter 30 for removing moisture from the breath sample as it is inputted. It should be noted that other forms of fluid or flow conditioner may be included in mouthpiece 18, examples of which could include without limitation such things as a flow restrictor, a pressure regulator, a flow truncator (e.g., such as those disclosed in commonly-assigned U.S. Pat. Appl. No. 62/247,778 (Dkt. No. INVOY.017PR)), and others.

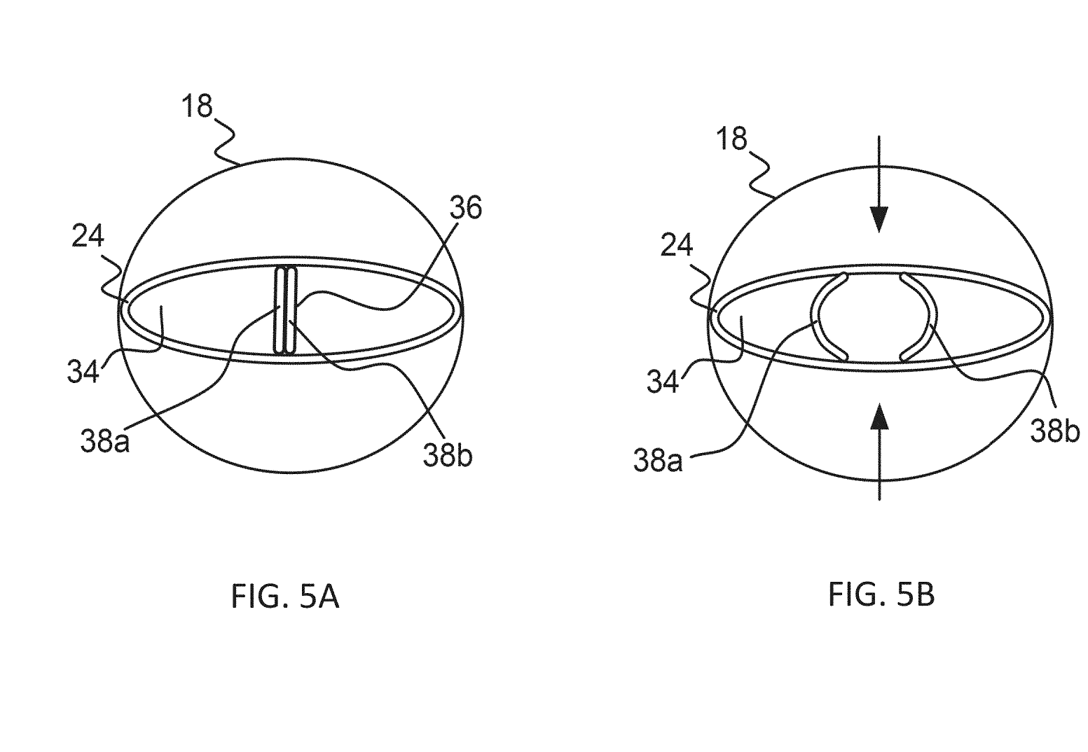

The input opening 22 of mouthpiece 18 may have a cap 32 to facilitate the sanitation of the mouthpiece and to prevent unwanted air, particles, objects, interferents, etc. from entering the interior portions of the mouthpiece. Alternatively, or in addition, for example, as shown in FIG. 4, a bite valve 34 may be provided within input opening 22. Bite valve 34 includes a resilient or elastomeric membrane 36, e.g., made of rubber, silicone or the like, with a slit valve 36 incorporated in its center. The slit valve comprises two adjacent and mating resilient flanges 38a and b that are moveable but normally biased closed. In this alternative embodiment, bite collar 24 is also resilient and is operatively coupled at top and bottom to flanges 38a and b. During normal conditions of non-use, slit valve 36 is biased in its closed position, as shown in FIG. 5, view A. In use, when the user bites the bite collar 24 in the directions shown by the arrows in FIG. 5, view B, vertical or compressive force is applied to flanges 38a and b, which forces them to bend outwardly, thereby creating an opening through which a breath sample may pass.

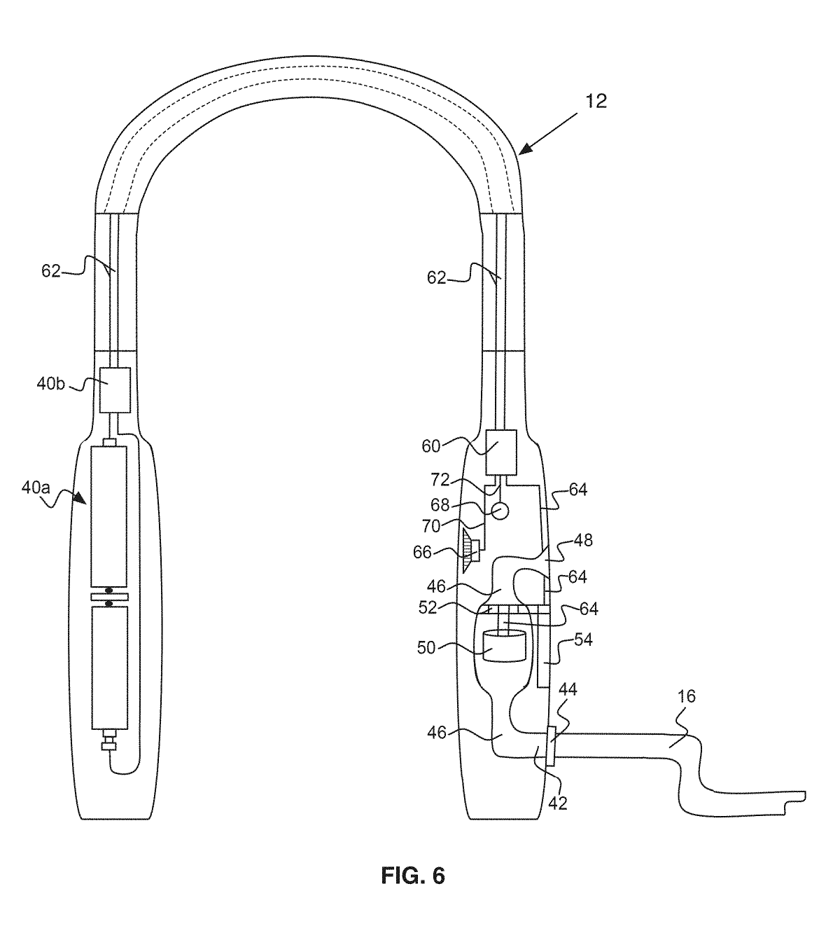

A partial cutaway side view of wearable 12 is shown in FIG. 6. Primary body half 12a comprises components associated with the breath analysis. Secondary body half 12b in this embodiment comprises a power supply 40, here in the form of a battery pack 40a, and, optionally, a power conditioner 40b for providing voltages as required by the device 12.

Turning to primary body half 12a, breath collection tube 16 enters body half 12a at an input conduit 42 via a coupler 44. Input conduit 42 extends into a main flow channel 46, which in turn extends into an exhaust port 48. An acetone sensor 50 is mounted to a support bracket 52 so that acetone sensor 50 is disposed in main flow channel 46 as air flows through the flow channel comprised of input conduit 42, main flow channel 46 and exhaust port 48. The acetone sensor may comprise any of a range of suitable sensors for this type of application. Examples include nanoparticle, enzyme-based, thermoelectric, quartz crystal microbalance, optical, colorimetric, metal oxide, semiconductor, magnetoelastic, and gravimetric sensors. Specific yet merely illustrative examples of such sensors include those disclosed in U.S. Pat. No. 6,609,068 (Dkt. No. INVOY.012C1), and U.S. patent application Ser. No. 11/656,338 (Dkt. No. INVOY.009P4), Ser. No. 13/052,963 (Dkt. No. INVOY.007A), Ser. No. 14/206,347 (Dkt. No. INVOY.003C1), and 61/593,862 (Dkt. No. INVOY.003PR), each of which is hereby incorporated herein by express reference as if fully set forth herein.

In some embodiments, acetone sensor 50 comprises a nanoparticle-based sensor, e.g., those described in commonly-assigned U.S. Pat. Appl. No. 62/161,872 (Dkt. No. INVOY.008PR). In view of the application for which this device 10 is configured, e.g., sensing breath acetone, an analyte sensor used in this embodiment may be a TGS 822 sensor, commercially available from Figaro USA, Inc. of Arlington Heights, Ill. But, other semiconductor or nanoparticle sensors may be used. An access door 54 is provided in the exterior housing of body half 12a to provide access to sensor 50, e.g., for testing, servicing, replacement, etc.

Primary body half 12a further houses a processor 60 which, for example, may comprise a commercially-available microprocessor or microcontroller capable of the configuration described herein and capable of performing the functions as described herein. Processor 60 is operatively coupled to power supply 40 to receive electrical power from it via a pair of leads 62. Processor 60 also is operatively coupled to analyte sensor 50 to receive the output of the sensor via leads 64.

Various breath input devices can be used in conjunction with the wearable. Initiation of the test may begin by a mechanical input (e.g., pushing a button), an auditory input (e.g., speaking to initiate the test), activation of a presence sensor (see, e.g., U.S. patent application Ser. No. 14/807,828 (Dkt. No. INVOY.002A4)), or other approaches. A bite valve may be used where the user bites a resilient mouthpiece to open a valve in the mouthpiece and then exhales into the tube to initiate the test. A breath input pressure valve may be used. Here, the test is initiated by the user exhaling into a tube. The valve is biased closed, but automatically opens when a threshold pressure is reached in the tube. In another embodiment, a breath input pressure sensor is used. The pressure sensor is disposed in the mouthpiece of the body of wearable and senses pressure. The test may be initiated when a threshold pressure is reached or exceeded.

The breath input devices may be passive or active. In an active input device, a pump may be used to extinguish the contents of a breath bag or breath container.

Device 10 also comprises at least one output device that outputs notifications or information to the user. The primary notification comprises the ketone body concentration measurements, or other information about those concentration measurements, for example, such as ratios of their values, time-dependent measurements, etc. The notifications and information may include such things as notifying the user of a scheduled analyte measurement test, steps and procedures to be used to initiate an analyte measurement test, steps and procedures to be taken during the course of a test, errors that occurred during testing, test results such as the measurement results of the test, and the like.

One may use a visual display to provide outputs. But, visual displays can be limiting, e.g., in that a display must be included in the device, the user must periodically consult the display by viewing it to check for notifications or information, etc. Moreover, particularly when the user is engaged in another activity, such as an exercise or work-out program, it may be inconvenient or difficult to visually inspect a display. Accordingly, the output comprises an output that notifies the user or gets his or her attention in a manner other than, or in addition to, a visual display. Examples of such output mechanisms include audio notification, e.g., by broadcasting, one or more tones, a tune or jingle, a speaking voice, etc., and/or a vibratory notification.

As implemented in some embodiments, device 10 comprises an audio output device 66, here comprising an amplifier and speaker for producing an audio tone, sequence of tones, a tune, a prerecorded speaking voice, etc. A vibratory device 68, for example, comprising an eccentric motor, also is provided in body half 12a for providing a vibratory signal or notification to the user. Audio output device 66 and vibratory device 68 are operatively coupled to and responsive to processor 60 via leads 70 and 72, respectively (they may also be operatively coupled to power supply 40 via suitable leads (not shown)).

Device 10 also comprises a blood ketone body measurement device 100 that provides a blood concentration measurement for at least one ketone body, e.g., at least one of acetoacetate and β-HBA. Measurement device 100 comprises an electromagnetic detection device 102 that is physically and electrically tethered or coupled to a cord 104. The other end of cord 104 is coupled to end 12b of wearable 12.

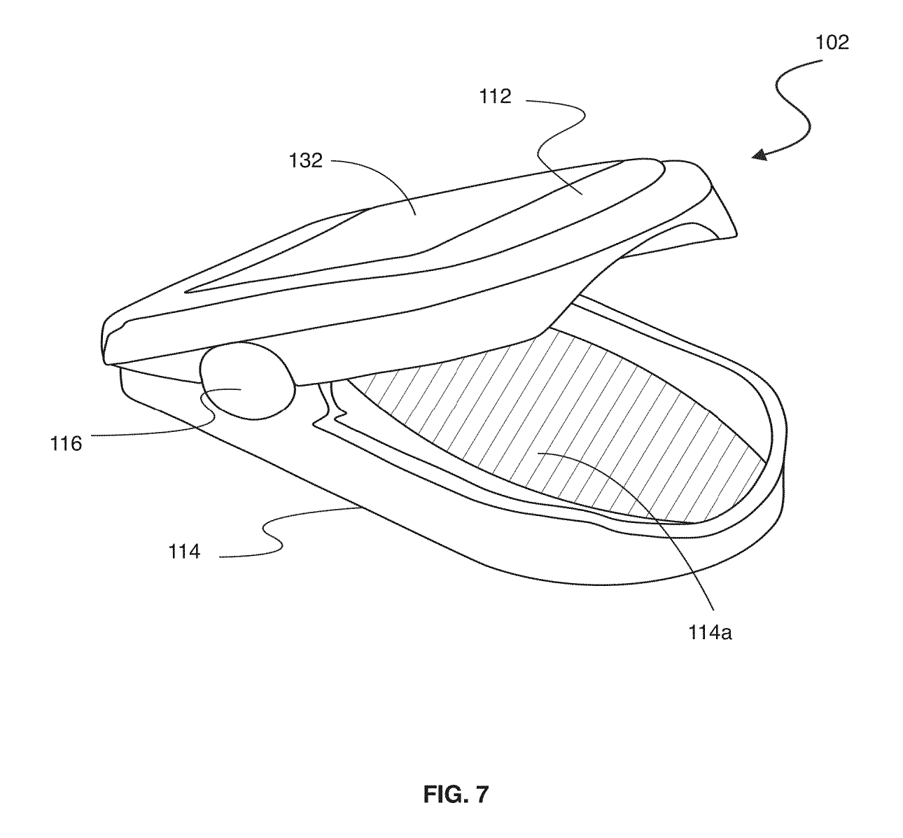

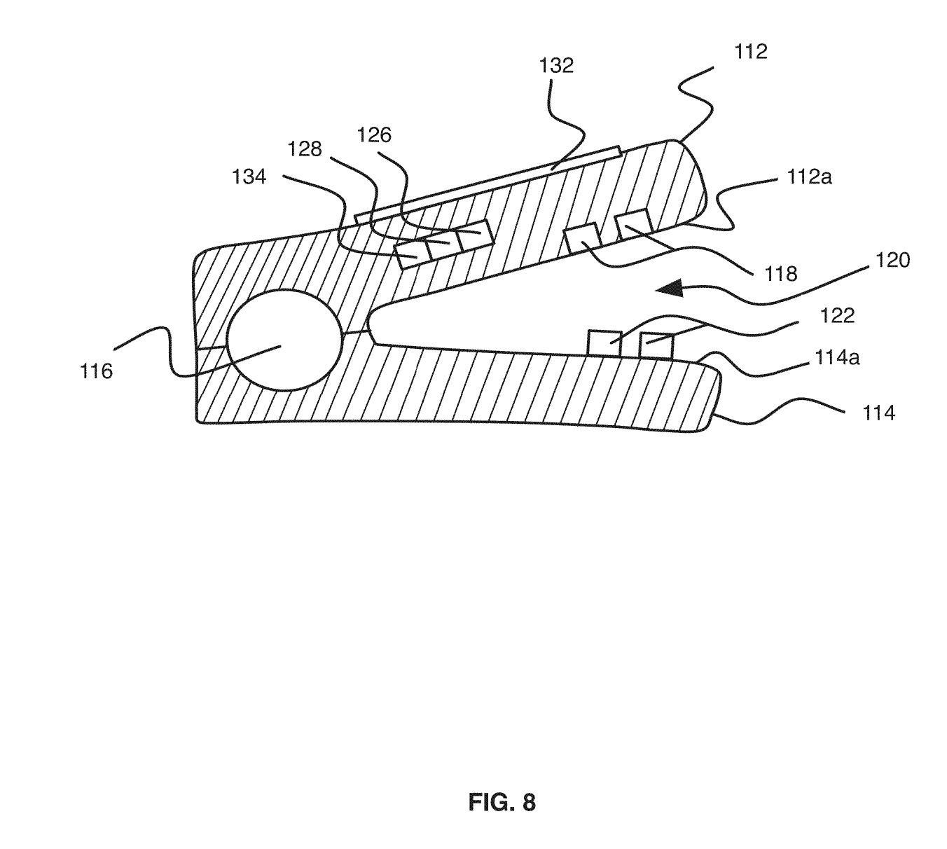

Electromagnetic detection device 102 is shown in perspective FIG. 7, and in side cross section in FIG. 8. Device 102 electromagnetically detects or measures the concentration of β-HBA in blood. The carbonyl group stretching vibration band of saturated aliphatic ketones occurs at 1715 cm¹. Conjugation of the carbonyl group with carbon-carbon double bonds or phenyl groups shift the band to wave numbers 1685-1666 cm⁻¹. The C—H bonds of alkyl groups stretch at about 2991 cm⁻¹. Thus, an EM detection device that operates within these wave number and corresponding frequency ranges can be used to detect and measure blood ketones, and specifically acetoacetate and/or β-HBA.

With reference to FIGS. 7 and 8, device 102 comprises an upper arm 112 and a lower arm 114 movably joined at a hinge 116. Upper arm 112 comprises an interior surface 112a and lower arm 114 comprises an interior surface 114a, each of which opposes the other. Hinge 116 is biased, e.g., using a spring (not shown), that urges the arms 112 and 114 and thus the interior surfaces 112a and 114a together.

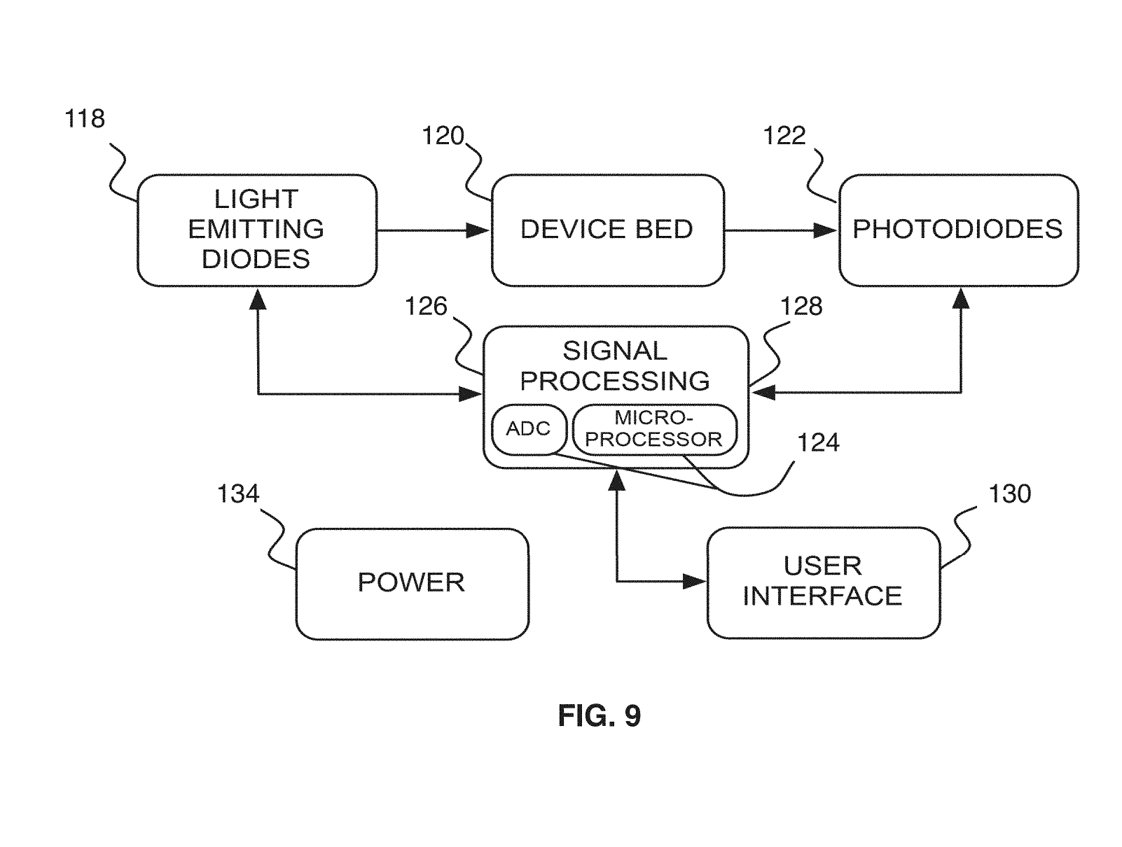

As further illustrated in FIG. 9, which is a block diagram of components of detection device 102, device 102 further comprises an electromagnetic (“EM”) illumination device, here in the form of a pair of light emitting diodes 118, a device bed 120 for finger placement and positioning, an EM sensing device, here in the form of a pair of photodiodes 122, and signal processor and output 124 comprising an AC to DC converter (“ADC”) 126, a microprocessor 128, and a user interface 130 comprising a display 132. Device 110 further comprises a power supply 134, which may comprise a battery pack, a power cord, or the like.

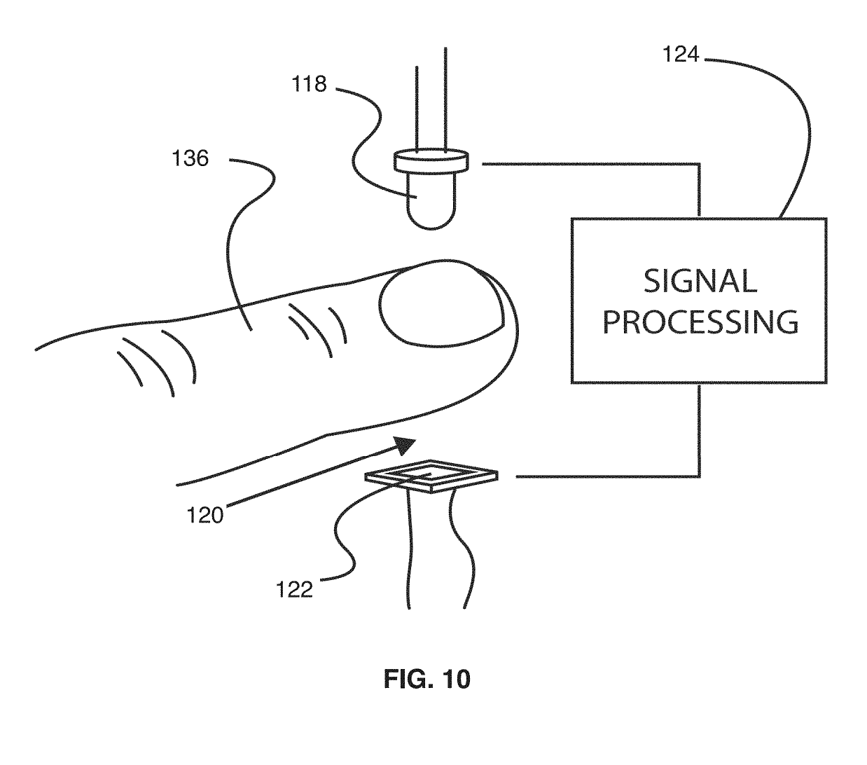

To better illustrate the arrangement and cooperation of these components, and with reference to FIG. 10, a finger 136 of the user is inserted into device bed 120 between upper and lower arms 112 and 114. Upon activation of device 102, which may comprise the user depressing a START or POWER button on upper arm 112 at or around display 132 (not shown), LEDs 118 illuminate downwardly toward and through finger 136. EM energy (e.g., light or infrared) is transmitted into and through the tissue of finger 136. A portion of that EM energy transmitted into the finger is absorbed by the molecular constituents of the finger, e.g., tissue, blood vessels and blood. The remainder passes out of the lower portion of the finger and impinges on photodiodes 122. Various portions of the absorbed light interact with and are absorbed by the respective constituents in the user's finger. The frequencies or wave lengths of the EM energy absorbed by a given molecular constituent are known, e.g., as described herein above for ketones. Signal processor 124, which receives a signal from photodiodes 122 indicative of the transmitted frequencies, can use the EM energy emanation information from LEDs 118 to ascertain the frequencies that have been absorbed. By comparing the amount of absorption at the frequencies characteristic of the analyte, signal processor 124 can thus ascertain a measure of the concentration of the analyte in the finger. In the case of device 102 as herein described, this yields a measure of the concentration of β-HBA in the blood within the user's finger 136.

Device 102 is configured such that the user's finger 136 is disposed in the device bed 120 so that the interior surfaces 112a and 114a of arms 112 and 114 are positioned substantially orthogonally with respect to the nail bed of finger 136. This is in contrast to virtually all commercially-available pulse oximeters, in which the interior surface of the top arm is substantially coplanar with respect to the nail bed of the user's finger.

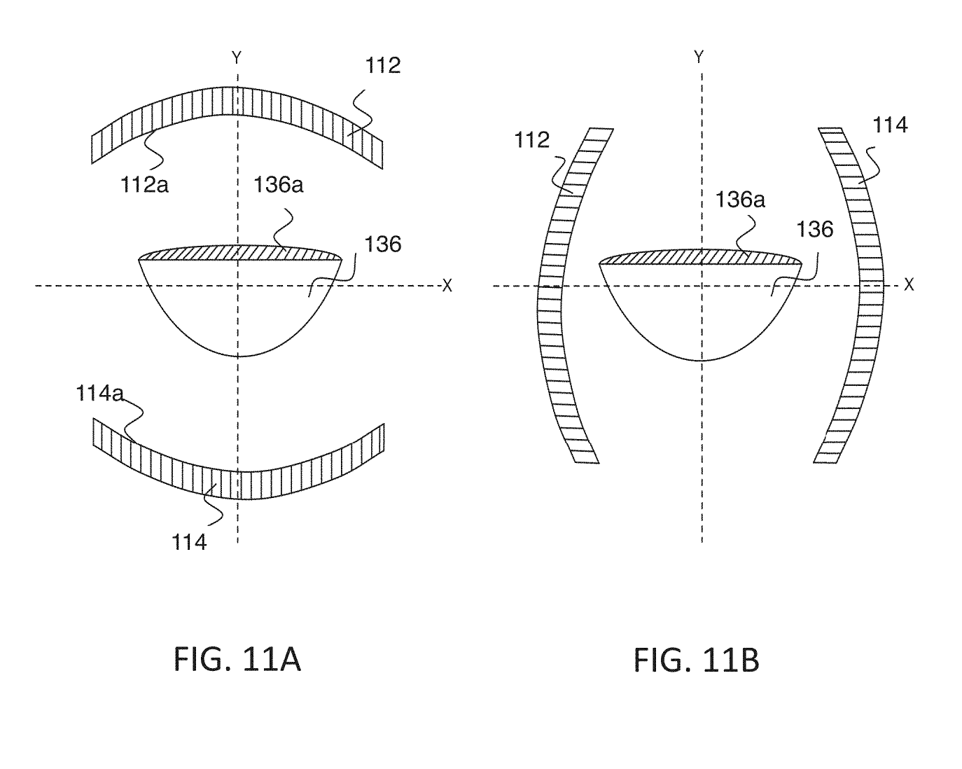

FIG. 11 illustrates, in view A, illustrates an end view of a commercially-available pulse oximeter having an upper arm PA-112 and lower arm PA-114. The arms are shown in cross section. The finger 136 of the user is disposed in such pulse oximeters such that the nail bed 136a of the user's finger 136 is adjacent to and contacts the interior surface PA-112a of the upper arm PA-112. With reference to the rectilinear x and y axes superposed on view A, the nail bed 136a lies on the y axis, which y axis extends through the upper and lower arm interior surfaces 112a and 114a.

In contrast, and as illustrated in view B of FIG. 11, in device 102 the user's finger 136 is disposed in the device bed 120 so that the nail bed 136a of finger 136 is substantially orthogonal with respect to the so that the interior surfaces 112a and 114a of arms 112 and 114 (shown in cross section in view B) are substantially orthogonally with respect to the nail bed of finger 136. With reference to the rectilinear axes, the nail bed lies in the x axis, which is perpendicular with respect to the y axis, the latter of which passes through the inner surfaces 112a and 114a.

As noted herein, there are many applications in which the EM detection system may need to be operable by and function for lay users. In such instances, there is a relatively enhanced likelihood of variability in placement of the device on the body from test to test, and potentially with inadvertent variability in placement from body part to body part. This is particularly true where there is a risk of interference from variable factors such as those described herein above, e.g., such as use of different fingers for the test, the presence of nail polish, and the like. This configuration passes the EM beam or illumination through the tissue in the sides of the finger, rather than through the nail and depth of the finger. The tension on the arms 112 and 114, e.g., as regulated by hinge 116, can apply sufficient force so that the interior surfaces 112a and 114a firmly contact and to a certain extent compress the finger so that surfaces 112a and 114a directly contact the outer surfaces of the finger substantially without air gaps. In some applications it is even desirable to physically compress the tissue in the figure.

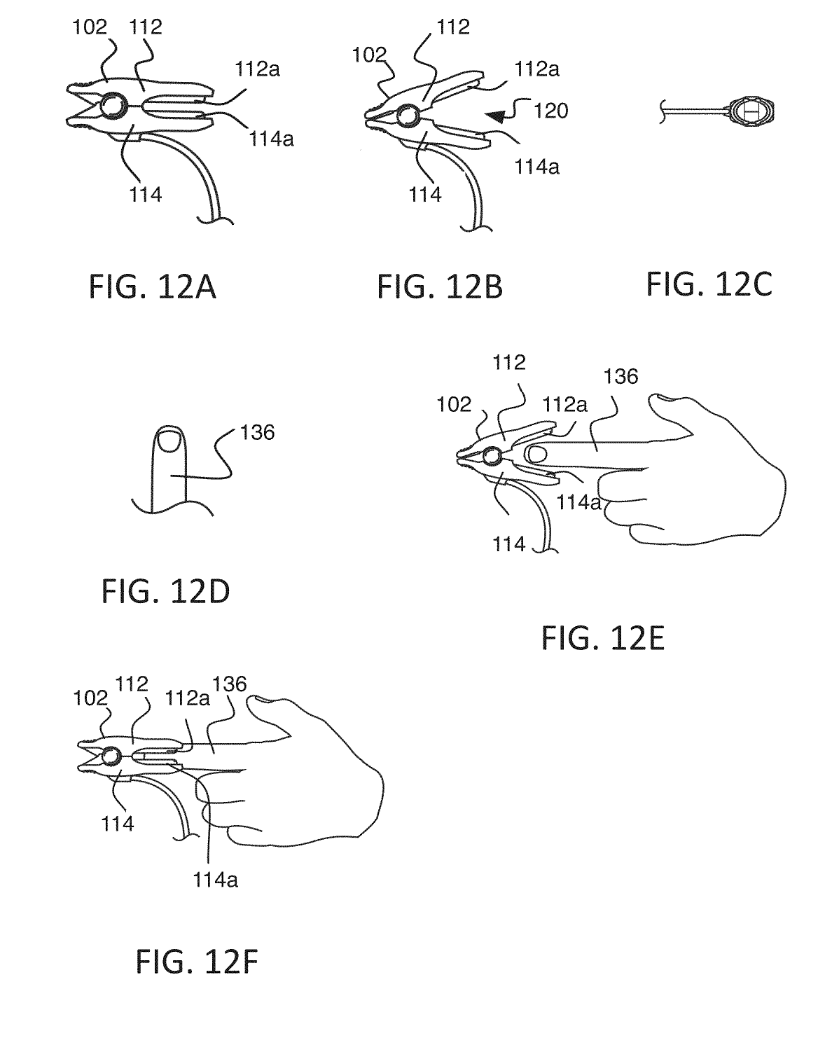

An illustration of the manner in which the finger of the user is inserted into device 102 in the course of using and operating device 102 to make an analyte measurement will now be described with reference to FIG. 12. View A shows a side view of device 102 with arms 112 and 114 in the closed position. In view B, the arms 112 and 114 have been biased open by applying pressure to the posterior end of the device adjacent to the hinge 116, thereby forcing the arms 112 and 114 open at the opposite or anterior end of the device to open access to the device bed 120. In this embodiment, the opening formed at the anterior end of device 120 is elliptical with the long axis corresponding to the x axis in FIG. 11, view B, to accommodate the elliptical dimensions of the user's finger 136, as illustrated in FIG. 12, views C and D. With reference to view E, with the arms 112 and 114 open the finger 136 is inserted into the anterior end of the device and into device bed 120. The force at the posterior end of device 102 may be released so that the biased arms 112 and 114 close to contact and grip the sides of finger 136. At this point, the device 102 is configured to energize LEDs 118 and conduct the analyte measurement.

Another embodiment of an EM device 102′ will now be described. This embodiment is very similar to device 102 in most respects, but in addition uses a custom user mold in the device bed to conform the interior arms of the device to the patient's finger.

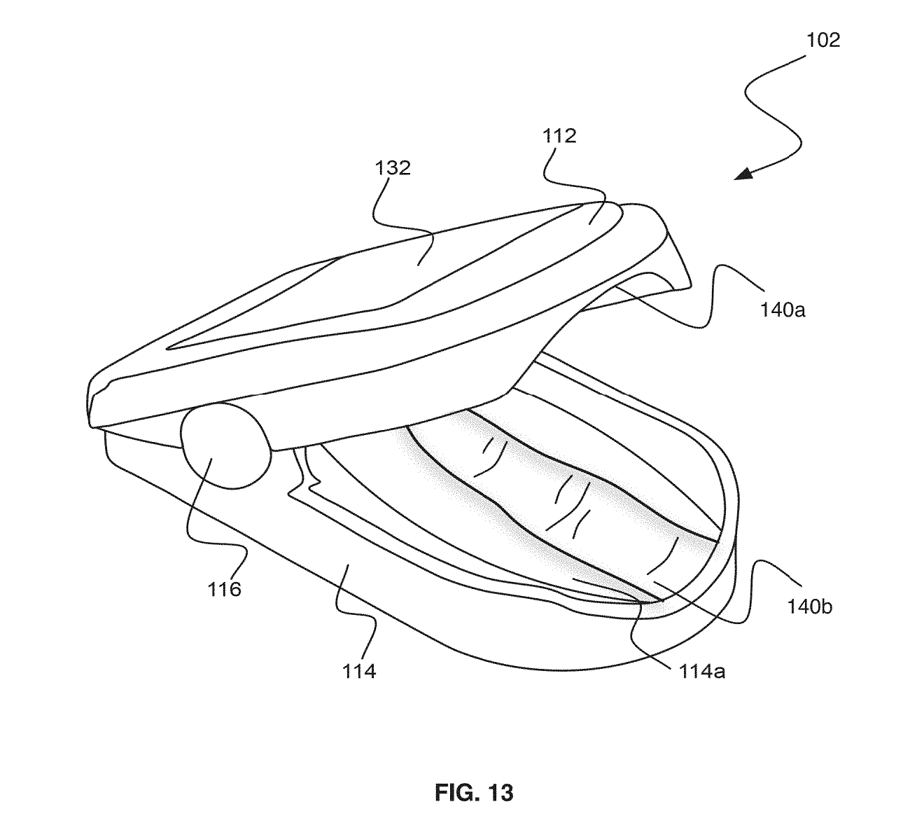

EM device 102′ is shown in perspective view in FIG. 13. As with device 102, device 102′ comprises upper arm 112 with interior surface 112a, lower arm 114 with interior surface 114a, and biased hinge 116 movably joining arms 112 and 114. Device 102′ also comprises LEDs 118, device bed 120, photodiodes 122, and signal processor and output 124 comprising ADC 126, microprocessor 28 and user interface 30 comprising display 32. Device 110 also includes power supply 34.

Device 102′ differs from device 102 in that device 102′ comprises a molded cavity 140 in device bed 120. Molded cavity 140 comprises an upper mold cavity 140a disposed in upper interior surface 112a and a lower cavity mold 140b disposed in lower interior surface 114a. Each of the upper and lower cavity molds is molded to and conformal with the sides of finger 136 of the user.

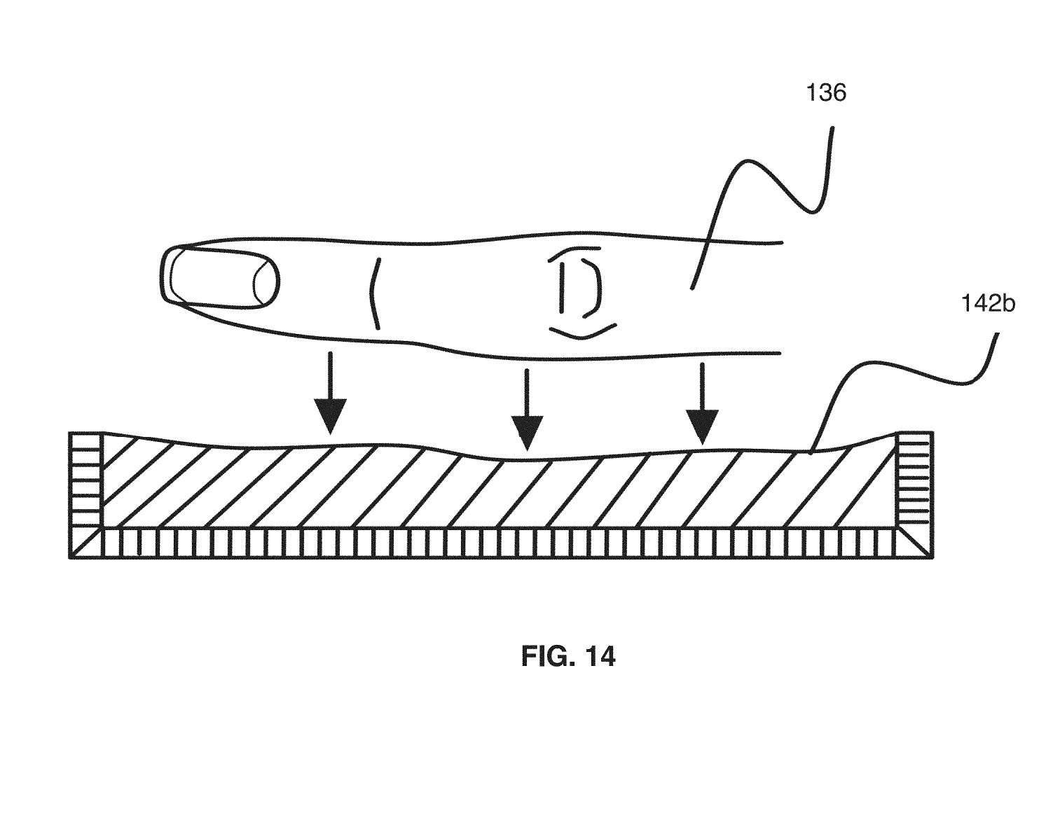

The molded cavity of device 102′ can be manufactured in the following manner. With reference to FIG. 14, a blank mold 142 is provided that includes two mold halves 142a and 142b. Mold halves 142a and 142b are slidable with respect to one another so that they can be moved apart and then back together. A moldable blank 144a is disposed on mold half 142a and a moldable blank 144b is disposed on mold half 142b. Moldable blanks 144a and 144b comprise a moldable material, for example, such as silicone, plastics with low shore hardness ratings, etc.

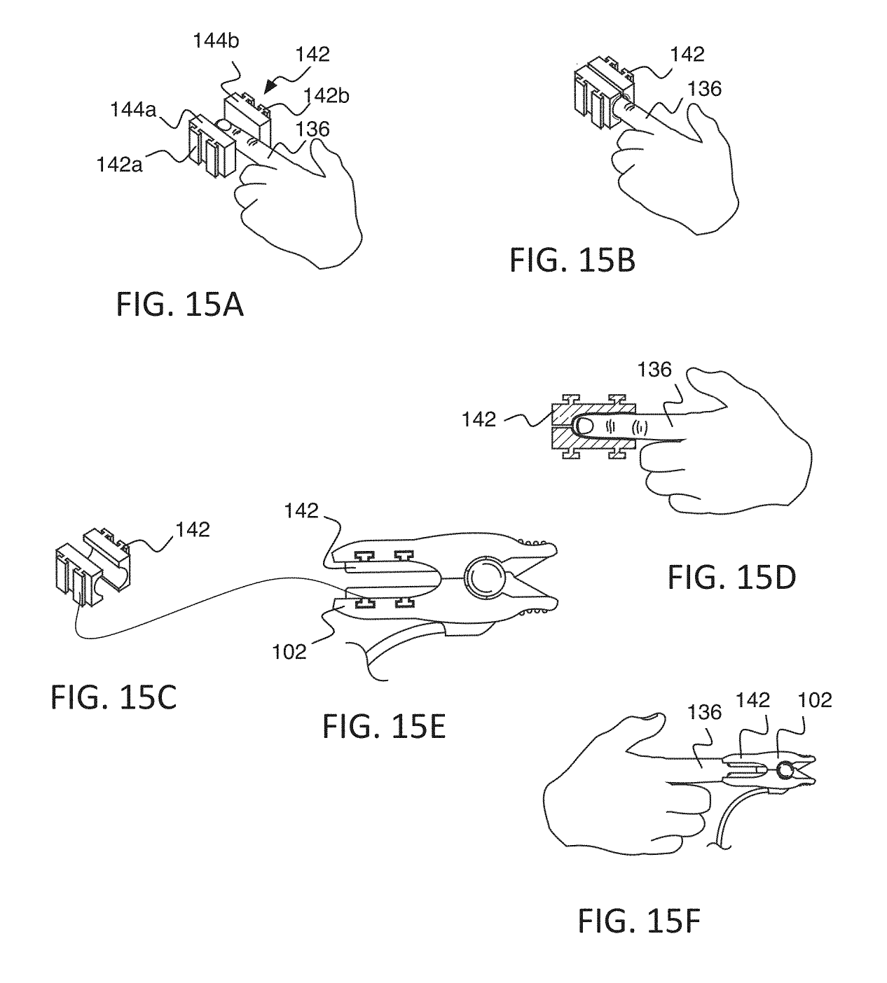

In making a custom mold for a user, the user inserts his or her finger into the open mold, as shown in view A of FIG. 15. The mold is then closed as shown in view B of FIG. 15 by moving the mold halves 142a and 142b together. As the moldable blanks contact the sides of the user's finger, the moldable blank material conforms to the contour of the finger. After a suitable curing time, the mold is opened and the user withdraws his or her finger. At this point, the moldable material has hardened or cured into a surface suitable for use as the equivalents of interior surface 112a and 114a of the device 102′. Holes or apertures are then drilled or pressed into the molded surface to accommodate the LEDs 118 and photodiodes 122. The mold halves are attached to interior surfaces 112a and 114a by suitable means, such as an adhesive. Fastening flanges also may be used (see, e.g., view E).

In use, the user inserts his or her finger into the anterior end of the open device and the arms are closed so that the molded material of mold halves 142a and 142b contact and conform to the sides of the user's finger 136. The LEDs are then energized to conduct the analyte analysis.

Returning to FIG. 1, device 10 further comprises an output device that receives the breath acetone concentration and the blood β-HBA concentration and presents information to the user. In device 10, the output device comprises a commercially-available mobile device 180 operating in conjunction with an application or “App” loaded on it. In addition to its role as an output device, mobile device 180 may serve as a command, control, and/or reporting device. It interacts with and controls measurement devices to conduct the breath analysis testing, but many of the control and reporting functions are configured in the mobile device instead of the wearable itself. It should be noted that one may modify either of the designs described herein for wearable 12 and output device 180 so that the various functionalities described herein are divided between the wearable and one or more external devices such as a mobile device.

Various algorithms and methods can be employed by the mobile device or the App. Examples of data visualization methods, signal processing algorithms, user interfaces and other methods are provided in U.S. patent application Ser. No. 14/807,828 (Dkt. No. INVOY.002A4) and Ser. No. 14/690,756 (Dkt. No. INVOY.006A), which are each incorporated herein by reference.

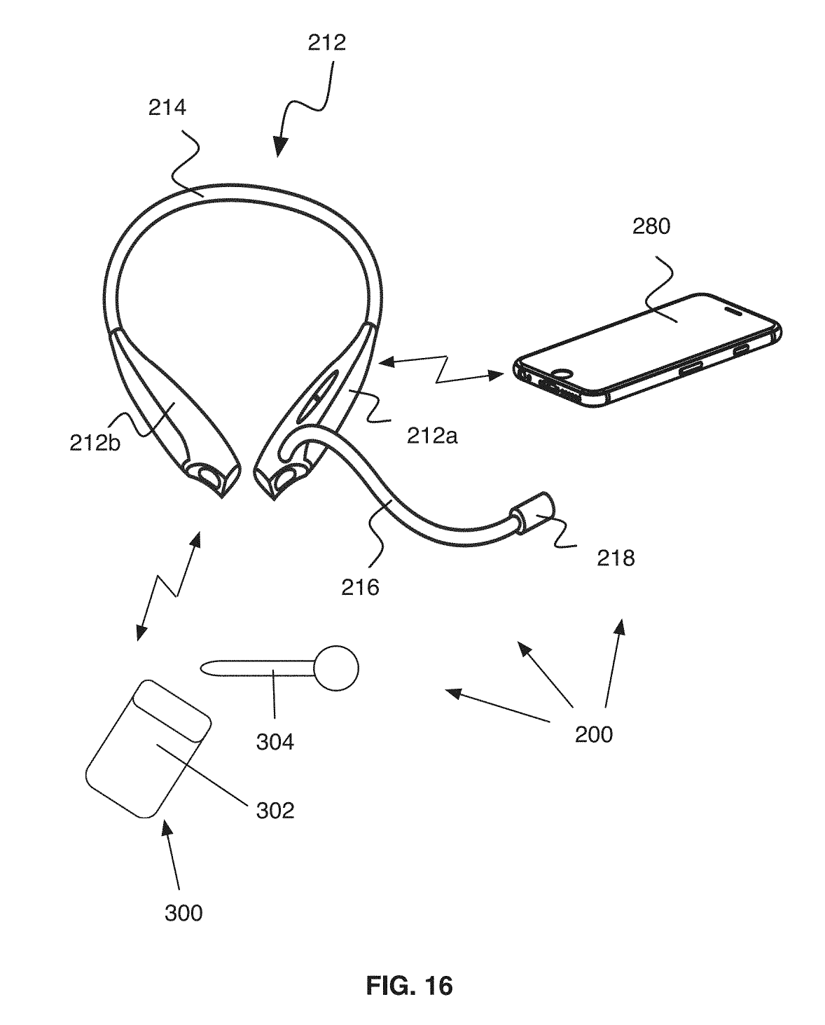

A device 200 according to another embodiment is shown in FIG. 16. Device 200 is similar or identical to device 10 in most respects, but differs in the embodiment of the blood ketone measurement device, using a manual blood measurement design rather than, or in addition to, an electromagnetic device design and previously shown and described.

Device 200 comprises a wearable 212 similar to wearable 12, that comprises band 2014 and body halves 212a and b, each similar to body 14 and body halves 12a and b, respectively. Device 200 also comprises an output device in the form of a mobile device 280 equivalent to mobile device 180. Command device 180 comprises a commercially-available mobile device, but also comprises a software application (“App”) or other suitable software or programming to carry out the functionality as described herein.

Device 200 comprises a blood ketone measurement device 300 that in turn comprises a measurement device body 302 and a detachable ketone test strip 304. Measurement device 300 may be any ketone measurement device that can perform the functions as described herein and meets general compatibility requirements of system 200. An example could be a commercially-available blood ketone measurement device, for example, such as Precision Xtra Device, however, includes a remote transmit feature such as an infrared, Bluetooth, etc. transmitter that can transmit blood ketone test results to mobile device 280.

In use, a user could use the breath acetone measurement device as described herein above. For measurement of blood-borne ketone bodies, the user could use a small lancet to produce a drop of blood on the user's finger in known fashion, use test strip 304 to absorb the blood droplet onto the test strip, and then insert the test strip into measurement device body 302. Measurement device 300 could then, e.g., in a known fashion, ascertain the blood ketone concentration measurement. The transmission device in measurement device 300 could then transmit this blood ketone concentration measurement to mobile device 280, whereupon device 280 could display it together with the breath acetone concentration measurement in desired form as described herein above.

Devices and methods according to the various embodiments disclosed herein may be used to assess compliance with a weight loss program and program effectiveness. This can be done, for example, by periodically measuring breath acetone and blood β-HBA.

### Example I

The following example pertains to a patient on a ketogenic diet for epilepsy management. Both plasma acetoacetate and β-HBA increase after the consumption of a ketogenic meal. Between meals, plasma acetoacetate but not β-HBA is directly metabolized for energy and, at other times, may also be used for fatty acid synthesis. In a sense, and in this situation, plasma β-HBA can be thought of as a ketone reserve—when plasma acetoacetate concentrations begin to decrease, more of it is produced from β-HBA.

Factors such as state of hydration and acid-base balance have complex effects on renal hemodynamics, urine volume, and excretion of ketone bodies and ultimately the assessment of the concentration of urinary acetoacetate.

### Example II

The following example pertains to alcohol ketoacidosis. Here, the interconversion of acetoacetate and beta-hydroxybutyrate occurs primarily in the mitochondria of liver cells. The generation of breath acetone, on the other hand, is from the non-enzymatic decarboxylation of acetoacetate occurring in the blood. The ratio of β-HBA to acetoacetate is dependent on the directionality of reaction catalyzed by β-HBA dehydrogenase enzyme. Under normal diet and health circumstances, the ratio is roughly 1:1 with a slightly higher proportion of β-HBA.

A factor (e.g., a major factor) that influences the ratio of β-HBA to acetoacetate and the underlying reactions is the ratio of nicotinamide adenine dinucleotide in its normal (NADH) and reduced (NAD⁺) states (NADH to NAD⁺). If more NADH is available, the reaction will favor β-HBA synthesis. The normal NAD⁺ to NADH ratio is about 10:1. Under certain circumstances, however, such as alcoholic ketoacidosis, there is a great increase in NADH due to alcohol metabolism, which significantly skews the ketone body ratio in favor of β-HBA.

Thus, in an alcohol abstention program, for example, the program may comprise requiring the program participants to periodically use device 10 to test for blood β-HBA and breath acetone. A baseline for the absolute concentrations of these ketone bodies is established during monitored abstention at the early stages of the program. Thereafter, the program participant takes periodic measurements of breath acetone and β-HBA, e.g., in twice-daily measurements. If the ratio of β-HBA concentrations to breath acetone concentrations increases significantly in a given measurement or contiguous set of measurements, the consumption of alcohol and thus non-compliance with the program is implicated.

### Example III

The following example pertains to diabetes. Diabetes is the most common pathological cause of elevated blood ketones. In diabetic ketoacidosis (“DKA”), high levels of ketones are produced in response to low insulin levels and high levels of counter-regulatory hormones. In acute DKA, the ketone body ratio of β-HBA to acetoacetate rises from a normal of about 1:1 to as high as 10:1.

In many instances, acetone and acetoacetate levels are well correlated to one another. In this example, acetone is used as a surrogate for acetoacetate. As such, a device that measures β-HBA in blood and acetone in breath can be used to determine the extent of the pH abnormality.

### Example IV

The following example pertains to a device 10, which can be used, e.g., as part of an exercise or physical training exercise, to assess whether the user is in an aerobic state, or has transitioned into an anaerobic state.

By way of background, muscle glycogen produces glucose, which produces pyruvate, which then reacts with oxygen to produce carbon dioxide and water and releases energy. If there is insufficient oxygen to meet energy demands (e.g., the exercise intensity exceeds the rate at which the cardiovascular system can supply muscles with oxygen), the pyruvate ferments into lactate. As muscle glycogen levels drop, fat metabolism is increased so that it can fuel the aerobic pathways. Aerobic exercise may be fueled by glycogen reserves, fat reserves, or a combination of both, depending on the intensity. Prolonged moderate-level aerobic exercise at a high VO2 max level (e.g., 65%) results in the maximum contribution of fat to the total energy expenditure.

A significant amount of the existing scientific literature regarding aerobic and anaerobic states are written for individuals who are not in a low carbohydrate state. For such individuals, the body has been adapted to primarily use fat as a substrate for metabolism.

Different muscles recruit different fibers and use different energy sources. As such, depending on the type, intensity and duration of exercise, different fuel sources, different muscle fibers and corresponding different byproducts are used. For example, certain tissues preferentially use β-HBA over acetoacetate.

A ketone monitoring system can operate as follows. A first ketone (for example, β-HBA) and a second ketone (for example, acetone) are measured at a first time. At this first time, the two ketones have a first relationship with regards to one another (e.g., the ratio of first ketone to second ketone>1). Then, at a second time, the two ketones have a second relationship with regards to one another (e.g., the ratio of first ketone to second ketone<=1). The relationship between the two ketones can thus be compared.

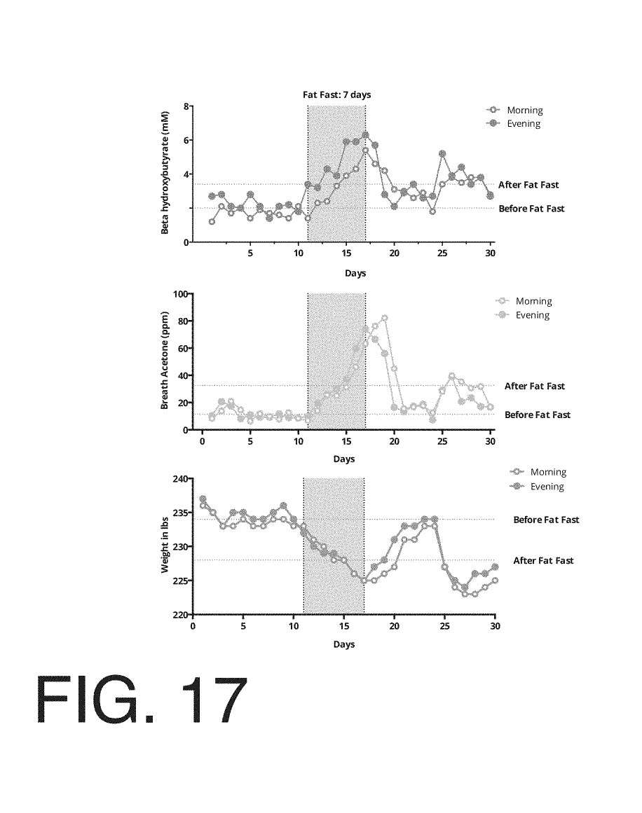

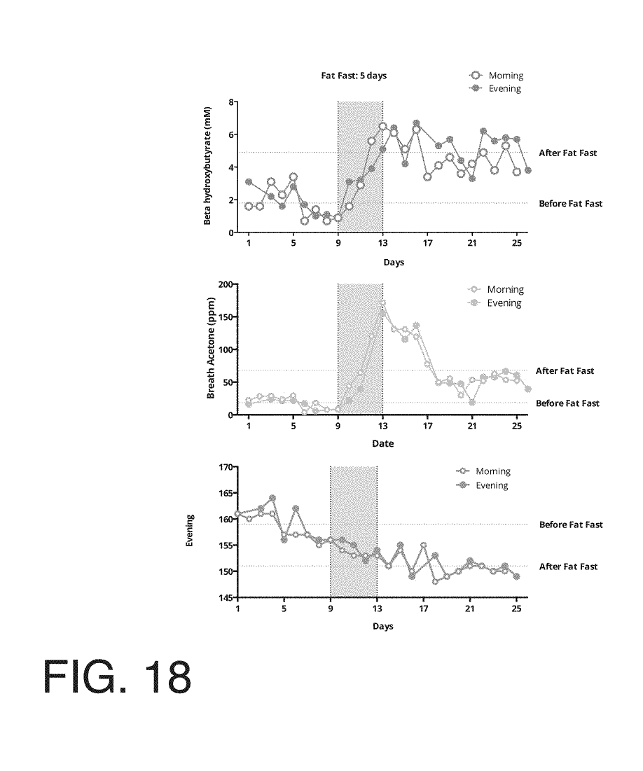

Examples of actual patient data measuring two ketones (breath acetone and blood β-HBA) are shown in FIG. 17 and FIG. 18.

In a situation in which a user employs two ketones to determine the transition from aerobic to anaerobic metabolism, the raw result of the two ketones may not be the target output. Rather, the device 10 may incorporate a signal, such as an LED that changes color, when the transition occurs. Other signals may be used, such as vibratory or auditory signals.

The desired times at which the two ketones can be measured depends on the application. In the case of exercise, some timing algorithms are shown in Table 1.

TABLE 1

| First Time | Second Time | Rationale |
|---|---|---|
| Before exercise | After exercise | Immediately post-exercise, |
| (30 minutes) | (<30 minutes) | for an individual who is low |
| | | carb adapted, ketone levels |
| | | are expected to decrease. |
| | | (Ketones are “consumed” or |
| | | used during the workout). |
| | | Depending on the type of |
| | | workout, the comparison |
| | | between the first and second |
| | | times may be quite different. |
| Before exercise | After exercise | At some point after exercise, |
| (30 minutes) | (>2 hours) | even for an individual who is |
| | | low carb adapted, ketone |
| | | levels will begin to increase |
| | | to their pre-workout time. |
| | | Depending on the type of |
| | | workout, the comparison |
| | | between the first and second |
| | | times may be quite different. |
| After exercise | After exercise | Comparing the rate of time |
| (<30 minutes) | (>2 hours) | for ketone levels to increase |
| During exercise | During exercise | Depending on the type of |
| (10 minutes) | (20 minutes) | workout, the relationship |
| | | between ketone levels may |
| | | change during the workout |
| | | itself. This change can be an |
| | | indicia of a transition from |
| | | aerobic to anaerobic |
| | | metabolism (or vice versa). |
| Before eating, | Before eating, | Even if an individual |
| before medication, | before medication, | was “low carb” at the |
| before exercise | before exercise | program start time, if the |
| (program start time) | (3 weeks after | individual begins to adhere |
| | program start time) | more strictly or incorporate |
| | | exercise, the relationship of |
| | | ketone levels can change |
| | | days later (in this example, 3 |
| | | weeks later) |

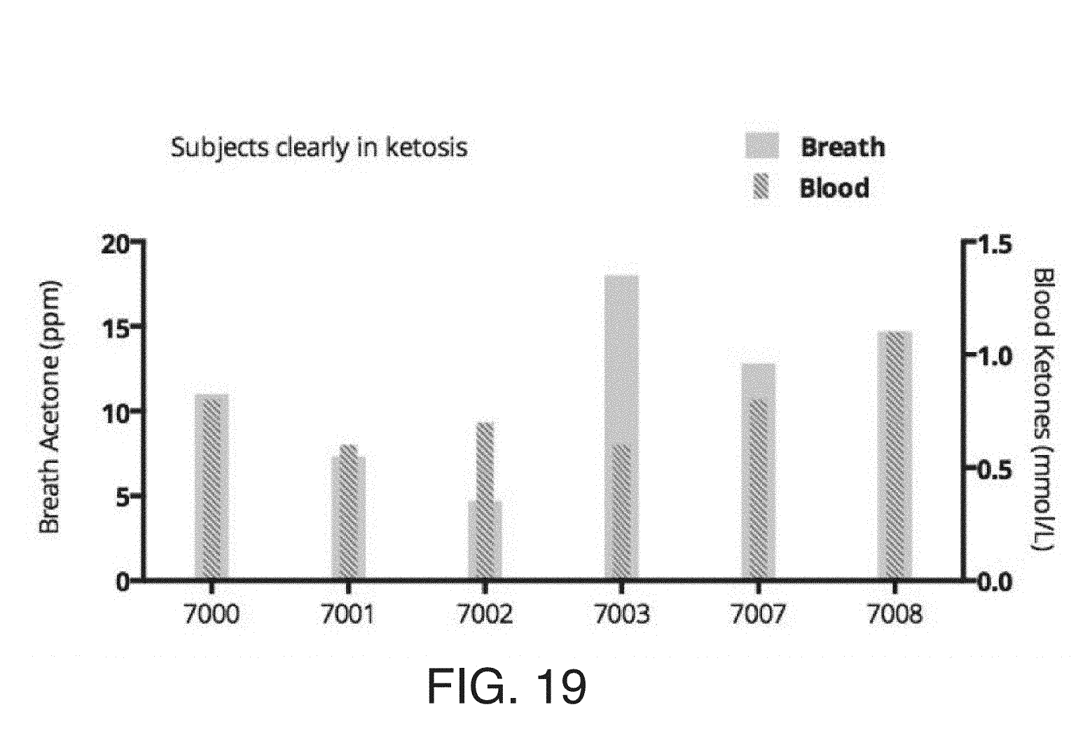

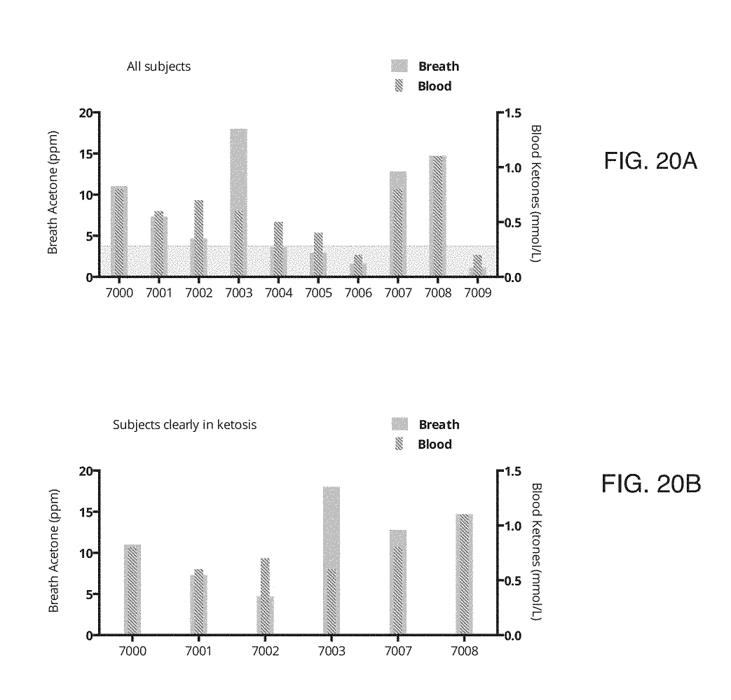

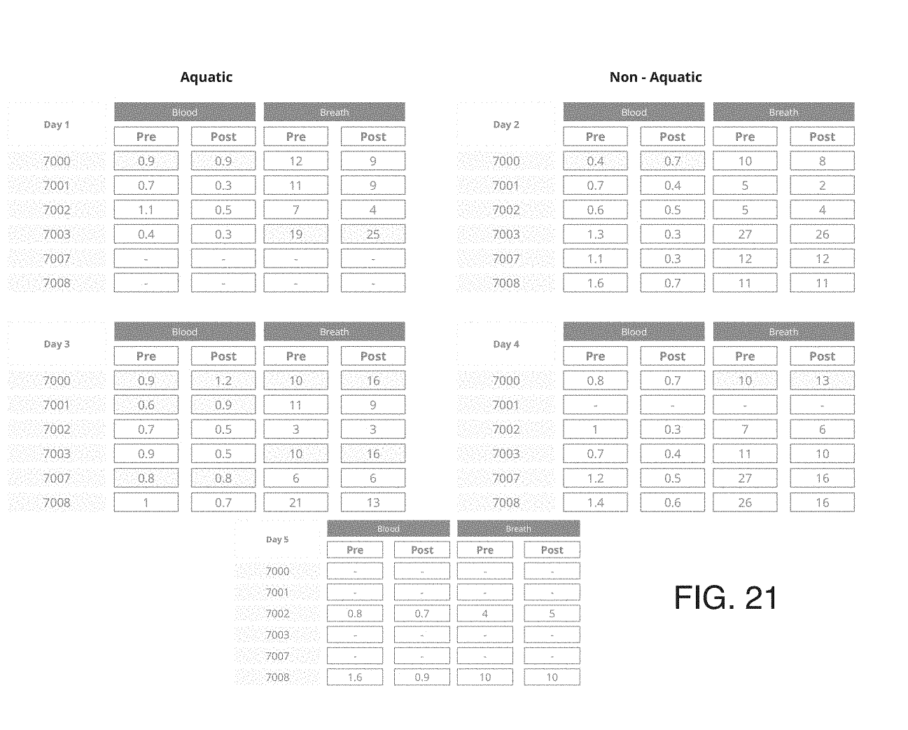

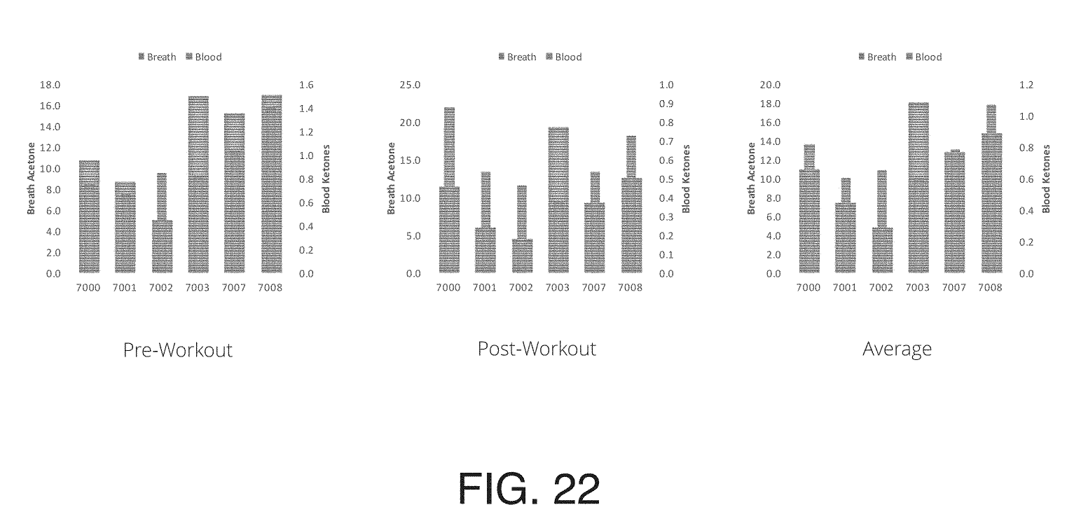

FIG. 19, FIG. 20 and FIG. 21 show pre and post exercise levels for different users. Note that, in this example, the ratio of a first ketone and a second ketone changes before and after exercise for the users. Additionally, in this example, the absolute values of certain ketones change before and after exercise. Also note that the nature of the change varies depending on the user's physiological state. In particular, user 7003 was more low carb adapted (e.g., had been on a low carbohydrate diet for a longer period of time) than other users.

This method of monitoring indicators of anaerobic and aerobic state has broader applicability. For example, instead of or in addition to using a second ketone, a different indicator of either aerobic or anaerobic metabolism can be used. For example, a combination device involving a ketone sensor and a lactic acid sensor may be employed. In such a situation, device 302 could be a lactic acid sensor.

The foregoing description and examples has been set forth merely to illustrate the disclosure and are not intended as being limiting. Each of the disclosed aspects and embodiments of the present disclosure may be considered individually or in combination with other aspects, embodiments, and variations of the disclosure. In addition, unless otherwise specified, none of the steps of the methods of the present disclosure are confined to any particular order of performance. Modifications of the disclosed embodiments incorporating the spirit and substance of the disclosure may occur to persons skilled in the art and such modifications are within the scope of the present disclosure. Furthermore, all references cited herein are incorporated by reference in their entirety.

Terms of orientation used herein, such as “top,” “bottom,” “horizontal,” “vertical,” “longitudinal,” “lateral,” and “end” are used in the context of the illustrated embodiment. However, the present disclosure should not be limited to the illustrated orientation. Indeed, other orientations are possible and are within the scope of this disclosure. Terms relating to circular shapes as used herein, such as diameter or radius, should be understood not to require perfect circular structures, but rather should be applied to any suitable structure with a cross-sectional region that can be measured from side-to-side. Terms relating to shapes generally, such as “circular” or “cylindrical” or “semi-circular” or “semi-cylindrical” or any related or similar terms, are not required to conform strictly to the mathematical definitions of circles or cylinders or other structures, but can encompass structures that are reasonably close approximations.

Conditional language used herein, such as, among others, “can,” “might,” “may,” “e.g.,” and the like, unless specifically stated otherwise, or otherwise understood within the context as used, is generally intended to convey that some embodiments include, while other embodiments do not include, certain features, elements, and/or states. Thus, such conditional language is not generally intended to imply that features, elements, blocks, and/or states are in any way required for one or more embodiments or that one or more embodiments necessarily include logic for deciding, with or without author input or prompting, whether these features, elements and/or states are included or are to be performed in any particular embodiment.

Conjunctive language, such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

The terms “approximately,” “about,” and “substantially” as used herein represent an amount close to the stated amount that still performs a desired function or achieves a desired result. For example, in some embodiments, as the context may dictate, the terms “approximately”, “about”, and “substantially” may refer to an amount that is within less than or equal to 10% of the stated amount. The term “generally” as used herein represents a value, amount, or characteristic that predominantly includes or tends toward a particular value, amount, or characteristic. As an example, in certain embodiments, as the context may dictate, the term “generally parallel” can refer to something that departs from exactly parallel by less than or equal to 20 degrees.

Unless otherwise explicitly stated, articles such as “a” or “an” should generally be interpreted to include one or more described items. Accordingly, phrases such as “a device configured to” are intended to include one or more recited devices. Such one or more recited devices can be collectively configured to carry out the stated recitations. For example, “a processor configured to carry out recitations A, B, and C” can include a first processor configured to carry out recitation A working in conjunction with a second processor configured to carry out recitations B and C.

The terms “comprising,” “including,” “having,” and the like are synonymous and are used inclusively, in an open-ended fashion, and do not exclude additional elements, features, acts, operations, and so forth. Likewise, the terms “some,” “certain,” and the like are synonymous and are used in an open-ended fashion. Also, the term “or” is used in its inclusive sense (and not in its exclusive sense) so that when used, for example, to connect a list of elements, the term “or” means one, some, or all of the elements in the list.

Overall, the language of the claims is to be interpreted broadly based on the language employed in the claims. The language of the claims is not to be limited to the non-exclusive embodiments and examples that are illustrated and described in this disclosure, or that are discussed during the prosecution of the application.

Although systems and methods of and for portable device for monitoring a plurality of ketones have been disclosed in the context of certain embodiments and examples, this disclosure extends beyond the specifically disclosed embodiments to other alternative embodiments and/or uses of the embodiments and certain modifications and equivalents thereof. Various features and aspects of the disclosed embodiments can be combined with or substituted for one another in order to form varying modes of systems and methods of and for portable device for monitoring a plurality of ketones. The scope of this disclosure should not be limited by the particular disclosed embodiments described herein.

Certain features that are described in this disclosure in the context of separate implementations can be implemented in combination in a single implementation. Conversely, various features that are described in the context of a single implementation can be implemented in multiple implementations separately or in any suitable subcombination. Although features may be described herein as acting in certain combinations, one or more features from a claimed combination can, in some cases, be excised from the combination, and the combination may be claimed as any subcombination or variation of any subcombination.

While the methods and devices described herein may be susceptible to various modifications and alternative forms, specific examples thereof have been shown in the drawings and are herein described in detail. It should be understood, however, that the invention is not to be limited to the particular forms or methods disclosed, but, to the contrary, the invention is to cover all modifications, equivalents, and alternatives falling within the spirit and scope of the various embodiments described and the appended claims. Further, the disclosure herein of any particular feature, aspect, method, property, characteristic, quality, attribute, element, or the like in connection with an embodiment can be used in all other embodiments set forth herein. Any methods disclosed herein need not be performed in the order recited. Depending on the embodiment, one or more acts, events, or functions of any of the algorithms, methods, or processes described herein can be performed in a different sequence, can be added, merged, or left out altogether (e.g., not all described acts or events are necessary for the practice of the algorithm). In some embodiments, acts or events can be performed concurrently, e.g., through multi-threaded processing, interrupt processing, or multiple processors or processor cores or on other parallel architectures, rather than sequentially. Further, no element, feature, block, or step, or group of elements, features, blocks, or steps, are necessary or indispensable to each embodiment. Additionally, all possible combinations, subcombinations, and rearrangements of systems, methods, features, elements, modules, blocks, and so forth are within the scope of this disclosure. The use of sequential, or time-ordered language, such as “then,” “next,” “after,” “subsequently,” and the like, unless specifically stated otherwise, or otherwise understood within the context as used, is generally intended to facilitate the flow of the text and is not intended to limit the sequence of operations performed. Thus, some embodiments may be performed using the sequence of operations described herein, while other embodiments may be performed following a different sequence of operations.

Moreover, while operations may be depicted in the drawings or described in the specification in a particular order, such operations need not be performed in the particular order shown or in sequential order, and all operations need not be performed, to achieve the desirable results. Other operations that are not depicted or described can be incorporated in the example methods and processes. For example, one or more additional operations can be performed before, after, simultaneously, or between any of the described operations. Further, the operations may be rearranged or reordered in other implementations. Also, the separation of various system components in the implementations described herein should not be understood as requiring such separation in all implementations, and it should be understood that the described components and systems can generally be integrated together in a single product or packaged into multiple products. Additionally, other implementations are within the scope of this disclosure.

Some embodiments have been described in connection with the accompanying figures. Certain figures are drawn and/or shown to scale, but such scale should not be limiting, since dimensions and proportions other than what are shown are contemplated and are within the scope of the embodiments disclosed herein. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, any methods described herein may be practiced using any device suitable for performing the recited steps.

The methods disclosed herein may include certain actions taken by a practitioner; however, the methods can also include any third-party instruction of those actions, either expressly or by implication. For example, actions such as “positioning an electrode” include “instructing positioning of an electrode.”

In summary, various embodiments and examples of systems and methods of and for portable device for monitoring a plurality of ketones have been disclosed. Although the systems and methods of and for portable device for monitoring a plurality of ketones have been disclosed in the context of those embodiments and examples, this disclosure extends beyond the specifically disclosed embodiments to other alternative embodiments and/or other uses of the embodiments, as well as to certain modifications and equivalents thereof. This disclosure expressly contemplates that various features and aspects of the disclosed embodiments can be combined with, or substituted for, one another. Thus, the scope of this disclosure should not be limited by the particular disclosed embodiments described herein, but should be determined only by a fair reading of the claims that follow.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example ±5%, ±10%, ±15%, etc.). For example, “about 1 V” includes “1 V.” Phrases preceded by a term such as “substantially” include the recited phrase and should be interpreted based on the circumstances (e.g., as much as reasonably possible under the circumstances). For example, “substantially perpendicular” includes “perpendicular.” Unless stated otherwise, all measurements are at standard conditions including temperature and pressure.

What is claimed is:

1. A device comprising:
a first ketone body measurement device that comprises a wireless transceiver and further measures a concentration of a first ketone body in a user;
a second ketone body measurement device different from the first ketone body measurement device that comprises a wireless transceiver and further measures a second ketone body different from the first ketone body in the user; and
an output device that receives the first and second ketone body concentration measurements and presents information to the user relating to the first and second ketone body concentration measurements.

2. A device comprising:
a breath acetone measurement device that measures a breath acetone concentration of a user;
a blood ketone body measurement device that measures at least one of a blood acetoacetate concentration and a blood β-HBA concentration of the user; and
an output device that receives the breath acetone concentration measurement and the at least one of the blood acetoacetate concentration and the blood β-HBA concentration and presents information to the user relating to these concentration measurements, optionally but preferably simultaneously.

3. A method comprising:
providing a portable device that comprises a first measurement device and a second measurement device different from the first measurement device;
using the first measurement device to obtain a concentration measurement of a first ketone body in a user;
using the second measurement device to obtain a concentration measurement of a second ketone body different from the first ketone body in the user; and
using the device to output information relating to the first and second ketone body concentration measurements, optionally but preferably simultaneously.

4. A method comprising:
providing a portable device that provides an output;
using the portable device to obtain a breath acetone concentration measurement of a user;
using the portable device to obtain a blood ketone body concentration measurement comprising at least one of an acetoacetate concentration and a β-HBA concentration of the user;
communicating the breath acetone concentration measurement and the blood ketone body concentration measurement to the output device; and
using the output device to provide information to the user.
